(19) United States
(12) Patent Application Publication
(10) Pub. No.: US 2021/0071227 A1
(43) Pub. Date: Mar. 11, 2021

(54) METHOD AND DEVICE FOR THE QUALITATIVE AND QUANTITATIVE DETECTION OF BIOFILM-BUILDING BACTERIA CONTAINED IN AN AQUATIC SYSTEM

(71) Applicant: Domatec GmbH, Mühldorf (DE)

(72) Inventors: Anna SALEK, Mühldorf (DE); Robert PRILLER, Mühldorf (DE)

(21) Appl. No.: 16/771,937

(22) PCT Filed: Oct. 31, 2018
(86) PCT No.: PCT/EP2018/079866

§ 371 Date: Jun. 11, 2020

(30) Foreign Application Priority Data
Dec. 11, 2017 (DE) 10 2017 222 366.9

(51) Int. Cl. C12Q 1/06; C12Q 1/10

(52) U.S. Cl. CPC C12Q 1/06; C12Q 1/10; G01N 2333/265; G01N 2333/245; G01N 2333/21

(57) ABSTRACT

A method and a device for performing are qualitative and quantitative assay of biofilm-forming bacteria of at least one bacterial species contained in an aquatic system by use of a biochemical reaction of the bacteria with at least one substance which initiates a bacterium-specific metabolic reaction, as well as a colorimetric reagent which can be influenced by the metabolic reaction involving measurement of the spectrometric properties thereof forming the basis of the assay.

### CROSS REFERENCE TO RELATED APPLICATION

Reference is made to PCT/EP2018/079866 filed Oct. 31, 2018, designating the United States, which claims priority to German Application No. 10 2017 222 366.9 filed Dec. 21, 2017, which are incorporated herein by reference in their entirety

### BACKGROUND OF THE INVENTION

### Field of the Invention

The invention relates to a method as well as to a device for the qualitative and quantitative assay of biofilm-forming bacteria of at least one bacterial genus contained in an aquatic system.

### Description of the Prior Art

Biofilms constitute microbial communities of cells which form in aquatic, that is aqueous systems, preferably on surfaces or interfaces of substrates in the form of a layer of slime formed by extracellular polymeric substances (EPS) produced by the microbial cells themselves. The layer of slime in this regard forms a kind of matrix in which the microbial cells, that is in particular of the genus Legionella spp., including the species Legionella pneumophila, are embedded, and in this manner are surrounded by a medium which enables the bacteria to live for lengthy periods.

Legionella spp. can occur both in fresh water and in salt water and have optimum conditions for life in the temperature range between 25° C. and 50° C. Thus, they are often found in equipment such as hot water production and distribution units, swimming pools, air washers in air conditioning units, cooling towers, dead legs, water tanks and the like. In particular, they can be found in hot water lines or cold water lines when heat acts on them from outside and when they have not been used for a long period of time.

In the past few years alone, many reports have been published regarding possible sources of the infection of people with Legionella pneumophila bacteria. In this regard, contaminated water distribution systems in buildings as well as industrial units, in particular cooling towers, constitute a particular threat to people. Thus, at the start of January 2010, a Legionella epidemic occurred in Germany, with 5 fatalities and 64 infected people, the cause of which could be traced to a cooling tower belonging to a cogeneration unit.

According to the provisions of the DVGW (Deutscher Verein des Gas-und Wasserfaches e. V.) [German Association for Gas and Water], Code of practice W 551, drinking water with a content of 100 cfu (colony forming units per 100 mL) is considered to be contaminated with a low risk of infection. Action is required beyond a value of more than 10000 cfu/100 mL, which makes it necessary to take immediate measures such as disinfecting the pipeline network and to impose a preliminary ban on use.

Yanez, M. A., et. al., in “Quantitative Detection of Legionella pneumophila in Water Samples by immunomagnetic purification and Real-Time PCR Amplification of the DotA Gene”, Applied and Environmental Microbiology, Vol. 71, No. 7, 07, pp. 3434-3436, describe a method for the quantitative determination of Legionella pneumophila in water samples by use of immunomagnetic purification and real-time PCR amplification of dotA genes.

Fuchslin et. al., in “Rapid and Quantitative Detection of Legionella pneumophila Applying Immunomagnetic Separation and Flow Cytometry”, Cytometry Part A, 77A, 03/2010, pp. 264-274, describe a method for the quantitative determination of Legionella pneumophila in water samples by use of a combination of immunomagnetic separation and flow cytometry.

The aforementioned analytical methods each require a sample to be taken in situ and detection in a laboratory equipped for the investigation of Legionella.

The published document WO 2011/157584 A1 discloses a portable device as well as a method for the qualitative and quantitative determination of Legionella in water. The known device operates in several stages, in which microorganisms contained in a defined quantity of a sample are separated by filtration and the Legionella are selectively separated from the separated microorganisms by immunomagnetic methods in order to subsequently be tagged using suitable fluorescence tags. The tagged Legionella are excited to fluorescence by use of a suitable excitation source and subsequently registered using a detector, and can be at least one of qualitatively and quantitatively evaluated via a computer system. In particular, the device is suitable for temporary or permanent integration into water transportation systems such as, for example, drinking water lines, cooling towers, air conditioning units, etc. Although the identification of Legionella in drinking water systems is already very advanced, evaporative cooling systems constitute a particular problem because of enhanced biofilm-forming accompanying flora such as, for example, Pseudomonas aeruginosa, Enterobacter aerogenes or Escherichia coli and the biofilm formation associated with it.

The article by Rahman, M. et al, The Catabolism of Arginine by Pseudomonas aeruginosa, J. Gen. Microbiol. (1980), 116, pp. 371-380, discusses the metabolism of mutations isolated from a strain of Pseudomonas aeruginosa which is not capable of using L-arginine or L-ornithine as the source of carbon for growth.

The article by Sabaeifard P., Abdi-Ali A., Soudi MR. und Dinarvand R., Optimization of tetrazolium salt assay for Pseudomonas aeruginosa biofilm using microtiter plate method, J. Microbiol. Methods, 2014 October, 105, pp. 134-140, concerns Pseudomonas aeruginosa, which is one of the most important pathogenic bacteria in connection with biofilm infections. Because of the multiple resistance of the biofilm, methods for counting biofilm formation are of interest in the assessment of an efficient drug regimen development for biofilm inhibition or extermination. When determining biofilm formation, vital or non-vital dyes are employed. The objective of the present study is the development of a test using a member of the tetrazolium salt family, 2,3,5-triphenyl tetrazolium chloride (TTC), for detecting Pseudomonas aeruginosa biofilm formation.

### SUMMARY OF THE INVENTION

The objective of the invention is to provide a method as well as a device for the qualitative and quantitative assay of biofilm-forming bacteria of at least one bacterial genus contained in an aquatic system in a manner such that a safe and rapid analysis and determination at least to detection of Legionella pneumophila is made possible within one or two days, preferably within 24 hours or less. In particular, it concerns a determination of Legionella pneumophila as well as other bacteria of different bacterial genuses within a multi-cellular community formed by biofilm-forming bacteria within an aquatic system in an accurate manner, in order to be able to take specific measures without delay for preventing or minimizing biofilm formation such as, for example, the use of bacterium-specific cleaning methods. The device in accordance with the solution should also be able to be used in situ, that is in the form of portable field equipment.

In contrast to known assaying methods for the detection of biofilm-forming bacteria which investigate the growth behavior (anabolism) of bacteria, and as a result take a lot of time to obtain a reliable prediction of the presence of appropriate bacteria, the method in accordance with the solution is based on the qualitative and quantitative assay of biofilm-forming bacteria of at least one bacterial genus contained in an aquatic system based on a biochemical reaction of the bacteria with at least one substance which initiates a bacterium-specific metabolic reaction by use of which a colorimetric reagent can be chemically influenced; its spectrometric or, more precisely, its spectrophotometric properties are measured and form the basis of the assay of the biofilm-forming bacteria contained in the aquatic system.

The method in accordance with the invention employs the measurement of the vitality or activity, based on catabolism, of specific biofilm-forming bacteria. In this regard, the metabolic reactions required to obtain the living cells are taken into consideration, wherein metabolic products of complex to simple molecules are broken down in order to detoxify the cell organism and produce energy. In this regard, the assay of bacteria of the bacterial genuses Legionella, Pseudomonas, Escherichia and Enterobacter are of particular interest.

Highly selective assay of bacteria in a specific bacterial genus within an aquatic system is possible with a substance that initiates a bacterium-specific metabolic reaction.

To assay bacteria of the bacterial genus Legionella, in particular of the bacterial species Legionella pneumophila, amino acids are advantageously suitable for the purposes of catabolization or metabolization by these bacteria, because amino acids, above all L-serine, L-threonine, constitute a preferred carbon and nitrogen source for Legionella pneumophila. In addition to the aforementioned amino acids, α-ketobutyric acid is also suitable, as a degradation product of the amino acid threonine, as well as pyruvic acid—for the purposes of catabolization via the bacterium Legionella pneumophila.

In order to determine the number of Legionella pneumophila bacteria within the aquatic system the metabolism of which is to be specifically excited or activated by at least one of the aforementioned substances, a colorimetric reagent is used, for example a tetrazolium salt, preferably in the form of 2,3,5-triphenyl-2H-tetrazolium chloride (TTC) or in the form of 2-(4-iodophenyl)-3-(4-nitrophenyl)-5-(2,4-disulphophenyl)-2H-tetrazolium, WST-1) with mPMS (1-methoxy-5-methylphenazinium methyl sulphate, as the electron mediator).

In addition, the assay known as the TTC or formazan test for the determination of the vitality of cells or bacteria, in particular of Legionella pneumophila, is particularly suitable for the metrological detection of the biofilm-forming bacteria contained in the aquatic system. The colorimetric reagent, which in the initial state is a colorless compound, is reduced by a chemical reaction with the living and thus metabolically active bacteria to a soluble formazan product the quantity of which is directly proportional to the number of living cells or bacteria within the aquatic system.

For the metrological detection of the spectrophotometric properties of the colorimetric reagent in the aquatic system, the optical spectrophotometry method is used, with which the optical extinction of the aquatic system colored by the color change of the colorimetric reagent is measured. In this regard, depending on the type of analysis, at least one of an absolute extinction value and an extinction function which varies with time can be determined in order, for example, to be able to detect the variation in the activity of the bacteria in the aquatic system which metabolize the substance with time. The rate of colour development thus provides information regarding at least one of the density and the catabolic activity of the bacteria within the aquatic system.

Because biofilm-forming bacteria, in particular Legionella pneumophila, catabolize amino acids, preferably L-serine and L-threonine or their degradation product a ketobutyric acid, preferably in a microaerophilic atmosphere, the biochemical reaction of the biofilm-forming bacteria contained in the aquatic system with the specifically selected substance as well as with the added colorimetric reagent is preferably carried out under microaerophilic incubation conditions at a temperature in the range between 25° C. and 45° C., preferably between 30° C. and 40° C., in particular at 37° C.

Reliable measurement results regarding the quantity, that is number, as well as the activity of the biofilm-forming bacteria present within the aquatic system can already be obtained after a reaction time between the bacteria and the respective bacterium-specific substance of approximately 20 to 50 hours, preferably even after 24 hours.

In addition to the qualitative as well as the quantitative assay of Legionella pneumophila within the aquatic system, the md in accordance with the solution is especially suitable for the further assay of bacteria of the following bacterial genuses: Pseudomonas, Escherichia, Enterobacter.

In order to assay bacteria of the bacterial genus Pseudomona, in particular the bacterium Pseudomonas aeruginosa, substances which are suitable for initiating the bacterium-specific metabolic reaction are L-arginine, L-asparagine, itaconic acid or putrescine.

In order to assay bacteria of the bacterial genus Enterobacter, in particular the bacterium Enterobacter aerogenes, substances which are suitable for initiating the bacterium-specific metabolic reaction are D-glutamic acid, D-cellobiose, β-methyl-D-glucoside or D-mannitol.

In order to assay bacteria of the bacterial genus Escherichia, in particular the bacterium Escherichia coli, substances which are suitable for initiating the bacterium-specific metabolic reaction are L-phenylalanine, α-D-glucose or glucose 1-phosphate.

At least a portion, preferably all of the aforementioned substances which are catabolized by the biofilm-forming bacteria, are used for the purposes of an assay of the biofilm-forming bacteria in an aquatic system which is as comprehensive as possible. To this end, the substances in question are each pre-loaded into mutually isolated cavities of a microtitre plate together with a colorimetric reagent as discussed above, into which cavities a sample of the aquatic system containing the biofilm-forming bacteria is introduced. [0028] The use of a microtitre plate allows for simple and above all individualized handling and carrying out of the biochemical reaction inside the individual cavities under uniform reaction conditions. The use of a microtitre plate which is transparent to light and which is known per se results in that after a specific reaction period has passed, a spectrophotometric measurement of all of the samples can be carried out within the individual cavities which have been isolated from each other.

The device in accordance with the invention for carrying out a qualitative and quantitative assay of biofilm-forming bacteria contained in an aquatic system in cavities each containing a substance and a colorimetric reagent is therefore characterized by a microtitre plate which is transparent to light, wherein in at least two cavities, a substance is contained which each are different from each other and which are selected from the group formed by the following substances: α-ketobutyric acid, L-serine, L-threonine, pyruvic acid methyl ester.

In a further preferred embodiment of the present microtitre plate designed for the purposes of assaying Legionella pneumophila, for a further assay of the bacteria Pseudomonas aeruginosa, in at least two further cavities, a substance is provided in each which respectively differ from each other and which are selected from the group formed by the following substances: L-arginine, L-asparagine, itaconic acid, putrescine.

Alternatively or in combination with both preferred exemplary embodiments as discussed above, in a further exemplary embodiment, the additional detection of the bacterium Enterobacter aerogenes is envisaged, in which in at least two further cavities, a substance is contained in each which differ from each other and which are selected from the group formed by the following substances: D-glutamic acid, D-cellobiose, β-methyl-D-glucoside, D-mannitol.

As an alternative or in combination with the preferred exemplary embodiments discussed above, in a final preferred exemplary embodiment, for the further assay of the bacterium Escherichia coli, in at least two further cavities, respectively different substances are provided which are selected from the group formed by the following substances: L-phenylalanine, α-D-glucose, glucose 1-phosphate.

In all of the exemplary embodiments discussed above for the formation of a microtitre plate in accordance with the invention, in addition to the aforementioned substances, it contains a colorimetric reagent, preferably in the form of a tetrazolium salt, preferably in the form of (2,3,5-triphenyl-2H-tetrazolium chloride, TTC) or (2-(4-iodophenyl)-3-(4-nitrophenyl)-5-(2,4-disulphophenyl)-2H-tetrazolium, WST-1) with mPMS (1-methoxy-5-methylphenazinium methyl sulphate, as the electron mediator).

A preferred use of the microtitre plate in accordance with the invention is for the qualitative and quantitative determination of Legionella pneumophila contained in aquatic systems, preferably of at least one further biofilm-forming bacterium of the following group: Pseudomonas aeruginosa, Enterobacter aerogenes, Escherichia coli.

The microtitre plate in accordance with the invention as well as the method in accordance with the invention associated therewith for the qualitative and quantitative assay of biofilm-forming bacteria is primarily suitable for the investigation of water in water distribution systems, that is comprising the source to the end user inclusively, both for the purposes of supplying to people and animals as well as in industrial units such as cooling towers, etc., for example.

Clearly, other uses for the investigation of aquatic systems regarding specific bacterial species may be envisaged, that is in particular in medical, chemical or biochemical fields, where appropriate fluids are to be analysed which constitute aquatic systems and which each constitute a medium for the survival of biofilm-forming bacteria. In the medical field in particular, the use of the microtitre plate in accordance with the invention is of great importance, in particular for the investigation of the following aquatic systems: bodily fluids, in particular urine, cerebrospinal fluid, bile, sputum, gastric juices, breast milk, vaginal secretions, lachrymal fluid, nasal discharges, ejaculate.

In a further advantageous embodiment for carrying out the assay method in accordance with the invention for the biofilm-forming bacteria which are present, which can all be assigned to what are known as the gram negative bacterial group, the bacteria to be assayed and which are contained in the aquatic system undergo at least one of a chemical and hydromechanical modification in which the cell membrane permeability of the bacteria is intentionally conditioned prior to at least one of before and during the biochemical reaction initiating the bacterium-specific metabolic process.

The at least one of chemically and hydromechanically initiated modification results in a variation in the cell surface properties and an associated increase in the permeability of the bacterial membrane.

In this regard, gram negative bacteria have non-specific transmembrane proteins, what are known as porins, in the respective outer membrane which act to exchange material through the membrane. It has, for example, been shown that by adding the surface-active chemical substance polysorbate 80 to the bacteria to be assayed, a significant increase in the permeability of the outer cell membrane of the bacteria can be obtained.

By use of the membrane modification which is undertaken, at least the electron or proton exchange through the outer membrane with the aquatic environment is boosted, whereupon the bacterium-specific metabolic reaction which can be initiated by the at least one substance can be carried out with a significantly higher efficiency and an associated temporal acceleration of the entire assay method. The increase in the physical permeability of the outer cell membrane brought about by the membrane modification also leads to an increase in the detection capability or an improvement in the sensorial sensitivity of the assay method in accordance with the invention. In this manner, small concentrations of bacteria in a sample can be assayed and in fact within very short assaying periods of significantly below 24 h.

In connection with the use of polysorbate 80, the bioenergetic activity, the temporal catabolic behavior of Legionella pneumophila, was measured by measuring the rates of electron flows in and through the electron transport chains with different metabolic substrates, for example of L-serine, L-threonine, α-ketobutyric acid and pyruvic acid methyl ester. Although each of the substances have different electron transport chains through the cell wall, the electrons will always migrate respectively from the proximal to the distal portion of the electron transport chain, where a redox dye acts as the terminal electron or proton acceptor, which upon reduction transforms the dye accordingly, and its change of color can be detected spectrophotometrically in the described manner.

As an alternative or in combination with the use of polysorbate 80 as a chemical agent that modifies the cell surface properties, bio-surfactants are also suitable for conditioning the cell membrane permeability of the bacteria. Rhamnolipids or surfactin are foremost in this regard.

One possibility for influencing the membrane permeability or for influencing the porins contained in the outer membrane of gram negative bacteria which is comparable to the aforementioned chemical measures the application of hydrodynamic forces on the bacteria contained in the aquatic system, namely by feeding ultrasound waves into the aquatic system. Experiments in this regard have shown that gram negative bacteria have an increased cell membrane permeability following the application of ultrasound for a period of approximately 15 minutes. A particularly advantageous effect is exhibited by the application of a combination of ultrasound with at least one of the chemical cell membrane conditioning measures discussed above to the respective gram negative bacteria to be assayed.

### BRIEF DESCRIPTION OF THE INVENTION

The invention will now be described by way of example in a manner which does not limit the general inventive concept, with the aid of an exemplary embodiment and with reference to the accompanying drawings, in which:

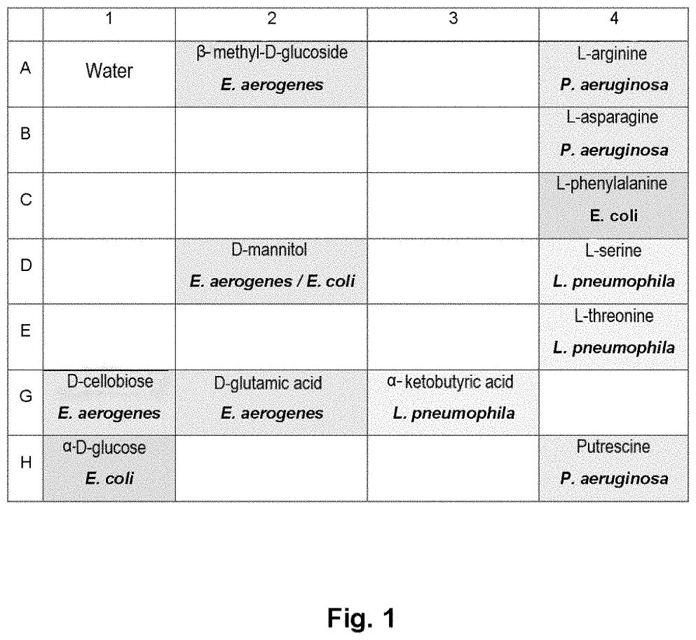

FIG. 1 shows a diagrammatic representation of the distribution of different substances in cavities of a microtitre plate.

### DETAILED DESCRIPTION OF THE INVENTION

FIG. 1 shows a tabular arrangement having four columns (1, 2, 3, 4) and eight rows (A-H), in which 32 fields can be distinguished from each other by an individual column and row address. The tabular arrangement constitutes a highly diagrammatic illustration of a microtitre plate the cavities of which are represented by the 32 fields.

Selected fields or cavities of a microtitre plate represented by the tabular arrangement each contain the substances that can be seen in FIG. 1, which can each initiate a bacterium-specific metabolic reaction. In each case, underneath the substance given in the individual fields/cavities, the name of the bacterium that reduces the substance in the aquatic system during the course of a metabolic reaction is shown in bold type. A representative example in this regard that may be cited is the cavity in row A column 2 which contains the substance β-methyl-D-glucoside in order to assay the bacterium Enterobacter aerogenes.

In addition to the twelve fields/cavities filled with different substances, in one cavity, preferably in the cavity A/1, water has been pre-loaded as a neutral buffer medium, with the aid of which in the context of a photometric measurement, a reference comparison with what is known as the blank sample can be undertaken.

Together with the substances pre-loaded in the cavities, each cavity contains a colorimetric reagent in the form of a redox indicator, preferably a tetrazolium salt, for example in the form of (2,3,5-triphenyl-2H-tetrazolium chloride, TTC) or (2-(4-iodophenyl)-3-(4-nitrophenyl)-5-(2,4-disulphophenyl)-2H-tetrazolium, WST-1) with mPMS (1-methoxy-5-methylphenazinium methyl sulphate, as the electron mediator).

The geometric arrangement illustrated in FIG. 1 of the individual substances distributed over the various cavities should not be understood to be limiting; clearly, almost any alternative spatial distributions of the substances in the cavities of a microtitre plate are possible. In addition, the fields/cavities which are not filled with substances, for example A/3, B/1, B/2, B/3, C/1 etc., could be filled with other substances or the substances which have already been disposed in the microtitre plate.

1.-25. (canceled)

26. A method for the qualitative and quantitative assay of biofilm-forming bacteria of at least one bacterial genus contained in an aquatic system by using of a biochemical reaction of the bacteria with at least one substance which initiates a bacterium-specific metabolic reaction, and a colorimetric reagent which is influenced by the metabolic reaction, comprising measuring spectrometric properties and using the measured properties in performing the assay.

27. The method as claimed in claim 26, wherein:
the bacteria contained in the aquatic system are assigned to one of the bacterial species of: Legionella pneumophila, Pseudomonas aeruginosa, Escherichia coli, Enterobacter aerogenes.

28. The method as claimed in claim 27, wherein:
the biochemical reaction for the assay of the bacteria of the bacterial genus Legionella, of the bacterium Legionella pneumophila, is carried out with at least one of substances: α-ketobutyric acid, L-serine, L-threonine, pyruvic acid methyl ester.

29. The method as claimed in claim 27, wherein:
the biochemical reaction for the assay of the bacteria of the bacterial genus Pseudomonas aeruginosa, is carried out with at least one of substances: L-arginine, L-asparagine, itaconic acid, putrescine.

30. The method as claimed in claim 28, wherein:
the biochemical reaction for the assay of the bacteria of the bacterial genus Pseudomonas aeruginosa, is carried out with at least one of substances: L-arginine, L-asparagine, itaconic acid, putrescine.

31. The method as claimed in claim 27, wherein:
the biochemical reaction for the assay of the bacteria of the bacterial genus of the bacterium Enterobacter aerogenes, is carried out with at least one of substances: D-glutamic acid, D-cellobiose, Q-methyl-D-glucoside, D-mannitol.

32. The method as claimed in claim 27, wherein:
the biochemical reaction for the assay of the bacteria of the bacterium Escherichia coli, is carried out with at least one of substances: D-mannitol, L-phenylalanine, α-D-glucose, glucose 1-phosphate.

33. The method as claimed in claim 26, wherein:
a redox indicator is used as the colorimetric reagent.

34. The method as claimed in claim 33, wherein:
a tetrazolium salt in the form of (2,3,5-triphenyl-2H-tetrazolium chloride, TTC) or in the form of (2-(4-iodophenyl)-3-(4-nitrophenyl)-5-(2,4-disulphophenyl)-2H-tetrazolium, WST-1) with mPMS (1-methoxy-5-methylphenazinium methyl sulphate, as the electron mediator) is used as the redox indicator.

35. The method as claimed in claim 26, wherein:
the spectrometric properties of the colorimetric reagent in the aquatic system are detected by use of optical spectrophotometry, in which an optical extinction which can be associated with an aquatic system containing bacteria of at least one bacterial genus is measured, in order to obtain at least one of an absolute extinction value and an extinction function which varies with time.

36. The method as claimed in claim 26, wherein:
the biochemical reaction is carried out under microaerophilic incubation conditions at a temperature in the range between 25° C. and 45° C.

37. The method as claimed in claim 36 wherein the temperature is 37° C.

38. The method as claimed in claim 26, wherein:
the spectrophotometric properties of the colorimetric reagent in the aquatic system are measured after a reaction period for the biochemical reaction of 20 hours to 50 hours.

39. The method as claimed in claim 38 wherein the measurement occurs after 24 hours.

40. The method as claimed in claim 26, wherein:
a microtitre plate with a mutually isolated cavities is used, in which one of the at least one substances as well as the colorimetric reagent is pre-loaded into each cavity, and a sample of the aquatic system containing the biofilm-forming bacteria of at least one bacterial genus is introduced into each of the cavities of the microtitre plate.

41. The method as claimed in claim 26, wherein:
at least one of prior to and while carrying out the biochemical reaction, the bacteria undergo at least one of a chemical and hydromechanical modification.

42. The method as claimed in claim 41, wherein:
the chemical modification is carried out by adding at least one chemical agent to the bacteria selected from agents: polysorbate 80, rhamnolipids, surfactin.

43. The method as claimed in claim 41, wherein:
the hydromechanical modification is carried out by applying ultrasound waves to the bacteria.

44. A microtitre plate for carrying out a qualitative and quantitative assay of biofilm-forming bacteria contained in an aquatic system, with cavities each containing a substance and a colorimetric reagent, wherein:
a different substance is contained in each of at least two cavities, the substances being selected from the group formed by the following substances: α-ketobutyric acid, L-serine, L-threonine, pyruvic acid methyl ester.

45. The microtitre plate as claimed in claim 44, wherein:
a different substance is contained in each of at least two further cavities, the substances being selected from the group formed by the substances: L-arginine, L-asparagine, itaconic acid, putrescine.

46. The microtitre plate as claimed in claim 44, wherein:
a different substance is contained in each of at least two further cavities, the substances being selected from the group formed by the following substances: D-glutamic acid, D-cellobiose, Q-methyl-D-glucoside, D-mannitol.

47. The microtitre plate as claimed in claim 44, wherein:
different substances are contained in each of at least two further cavities, the substances being selected from the group formed by the following substances: and α-D-glucose.

48. The microtitre plate as claimed in claim 44, wherein:
the cavities contain, as the colorimetric reagent, a redox indicator in the form of a tetrazolium salt (2,3,5-triphenyl-2H-tetrazolium chloride, TTC) or (2-(4-iodophenyl)-3-(4-nitrophenyl)-5-(2,4-disulphophenyl)-2H-tetrazolium, WST-1) with mPMS (1-methoxy-5-methylphenazinium methyl sulphate, as the electron mediator).

49. A use of the microtitre plate as claimed in claim 44, for performing a qualitative and a quantitative assay of Legionella pneumophila contained in an aquatic system.

50. A use of the microtitre plate as claimed in claim 48, for the qualitative and quantitative assay of biofilm bacteria, which contain at least one bacterial species of bacterial species in an aquatic system: Legionella pneumophila, Pseudomonas aeruginosa, Enterobacter aerogenes, Escherichia coli.

51. A use as claimed in claim 49, of investigating water as the aquatic system be removed from water distribution systems for people and animals, industrial units and cooling towers.

52. A use as claimed in claim 49 of investigating in medical, chemical or biochemical fields liquids as the aquatic system.

53. Use as claimed in claim 52, wherein:
An investigation is performed in the medical field, involving urine, cerebrospinal fluid, bile, sputum, gastric juices, breast milk, vaginal secretions, lachrymal fluid, nasal discharges, and ejaculate.
